(19) United States
(12) Patent Application Publication
(10) Pub. No.: US 2013/0150754 A1
(43) Pub. Date: Jun. 13, 2013

(54) ELECTRONIC STETHOSCOPES WITH USER SELECTABLE DIGITAL FILTERS

(71) Applicant: 3M INNOVATIVE PROPERTIES COMPANY, ST. PAUL, MN (US)

(72) Inventors: Daniel J. Rogers, Grant, MN (US); Raymond L. Watrous, St. Paul, MN (US); Craig D. Oster, Oakdale, MN (US); Daniel R. McIntyre, St. Paul, MN (US); Bjorn K. Andersen, Struer (DK)

(73) Assignee: 3M INNOVATIVE PROPERTIES COMPANY, ST. PAUL, MN (US)

(21) Appl. No.: 13/706,441

(22) Filed: Dec. 6, 2012

Related U.S. Application Data
(60) Provisional application No. 61/568,411, filed on Dec. 8, 2011.

(51) Int. Cl. A61B 7/04

(52) U.S. Cl. CPC A61B 7/04
USPC 600/586

(57) ABSTRACT

In one aspect, an auscultation system utilizes a digital filter to obtain a sound profile similar to a sound profile of a mechanical stethoscope. In another aspect, an auscultation system utilizes a digital filter to provide diagnostic filtering. In yet another aspect, an auscultation system utilizes a digital filter to compensate for a characteristic frequency response of a headset.

### BACKGROUND

Mechanical stethoscopes have been developed to detect sounds produced by the body, such as heart and lung sounds. The stethoscope, for example, is a fundamental tool used in the diagnosis of diseases and conditions of the cardiovascular system. It serves as the most commonly employed technique for diagnosis of such diseases and conditions in primary health care and in circumstances where sophisticated medical equipment is not available, such as in remote areas.

Clinicians readily appreciate that detecting relevant cardiac symptoms and forming a diagnosis based on sounds heard through the stethoscope. It is a skill that can take years to acquire and refine. The task of acoustically detecting abnormal cardiac activity is complicated by the fact that heart sounds are often separated from one another by very short periods of time, and that signals characterizing cardiac disorders are often less audible than normal heart sounds. Physicians have often invested considerable time memorizing the characteristics of normal and abnormal body sounds, for example, heart and lung sounds, heard with their particular stethoscope. For example, heart murmurs are graded depending on the characteristic loudness of sounds.

### SUMMARY

At least one aspect of the present disclosure features an auscultation system comprising a sensor, a digital filter and a user interface. The sensor is configured to detect acoustic signals from human body and generate medical measurement signals based on the detected acoustic signals. The digital filter is capable of filtering the medical measurement signals with a plurality of stethoscope simulation filters, each stethoscope simulation filter operable to provide a transfer function, the transfer function resulting in a frequency response of the auscultation system that is psychoacoustically equivalent to a frequency response of a mechanical stethoscope. The user interface is configured to accept input from a user, wherein the user interface is further configured to allow a user to select a stethoscope simulation filter from the plurality of stethoscope simulation filters and the digital filter is configured to filter the medical measurement signals with the selected stethoscope simulation filter.

At least one aspect of the present disclosure features an auscultation system comprising a sensor, a digital filter, and a user interface. The sensor is configured to detect acoustic signals from human body and generate medical measurement signals based on the detected acoustic signals. The digital filter is capable of filtering the medical measurement signals with one or more headset filters, each headset filter operable to provide a transfer function to compensate for a characteristic frequency response of a particular headset. The user interface is configured to accept input from a user, wherein the user interface is further configured to allow a user to select a headset filter from the one or more headset filters and the digital filter is configured to filter the medical measurement signals with the selected headset filter.

At least one aspect of the present disclosure features an auscultation system comprising a sensor, a digital filter, and a user interface. The sensor is configured to detect acoustic signals from human body and generate medical measurement signals based on the detected acoustic signals. The digital filter is capable of filtering the medical measurement signals with one or more diagnostic filters, each diagnostic filter operable to provide a band-pass filter to filter the medical measurement signals at one or more frequency ranges appropriate to a particular diagnosis. The user interface is configured to accept input from a user, wherein the user interface is further configured to allow a user to select a diagnostic filter from the one or more diagnostic filters and the digital filter is configured to filter the medical measurement signals with the selected diagnostic filter.

### BRIEF DESCRIPTION OF THE DRAWINGS

The accompanying drawings are incorporated in and constitute a part of this specification and, together with the description, explain the advantages and principles of the invention. In the drawings,

FIG. 1 illustrates human organ sounds range in terms of ⅓ octave frequency bands;

FIG. 2 illustrates a block diagram of an exemplary embodiment of an auscultation system;

FIGS. 3A and 3B illustrate exemplary system diagrams of auscultation systems;

FIG. 4 illustrates a transfer function of an exemplary mechanical stethoscope;

FIG. 5 illustrates an exemplary logical flowchart of an embodiment of designing a stethoscope simulation filter;

FIG. 6A illustrates a schematic view of an acoustic test system to test frequency response of a stethoscope;

FIG. 6B illustrates an exemplary user interface for filter selection;

FIG. 7 illustrates is a perspective view of one embodiment of an electronic stethoscope;

FIG. 8 illustrates an exemplary embodiment of an auscultation system adapted for telemedicine applications;

FIG. 9 illustrates an exemplary logical flowchart of an embodiment of designing a headset filter; and

FIG. 10 illustrates some exemplary pathologies.

### DETAILED DESCRIPTION

Although electronic stethoscopes have been in the markets for more than ten years, the sound profiles of electronic stethoscopes are often different from the sound profiles of mechanical stethoscopes. For example, a trained physician may distinguish the sound profile of a diaphragm filter of a LITTMANN Electronic Stethoscope Model 3100 from the sound profile of a LITTMANN Cardiology II S.E. This disclosure is directed to electronic stethoscope devices and systems using digital filtering to obtain sound profiles simulating the sound profiles of selected mechanical stethoscopes. Particularly, this disclosure is directed to electrical stethoscope devices and systems using digital filters having transfer functions simulating the frequency response of mechanical stethoscopes. The frequency response of a stethoscope refers to a mathematical representation of the input-to-output relation in terms of temporal frequency. Various embodiments of this invention use digital filters to provide desired transfer functions. Changes in one or more coefficients of a digital filter often are used to modify the corresponding transfer function.

In a variety of situations a user may use a headset with a stethoscope. For example, a user may use a separate headset in an ambulance, helicopter, a telemedicine facility, or other situations. When an auscultation system is used in telemedicine, a remote user may use a headset with accompanied receiving device and/or processing device to listen to auscultation signals measured from a patient. Headsets typically have characteristic frequency responses affecting the output signals of the auscultation system. For example, a headset may have some level of acoustic distortion. Systems and methods of the present disclosure are also directed to an auscultation system that includes a digital filter capable of filtering signals with one or more headset filters, where each headset filter is operable to provide a transfer function to compensate for frequency response characteristic of a particular headset to provide the desired stethoscope frequency response.

Because of the numerous body sounds that can be detected by an auscultation system, sometimes it is beneficial to allow a user to make a selection to focus on a particular diagnosis. A diagnosis selection is often related to one or more frequency ranges. For example, specific valvular and congenital lesions may produce heart sounds and murmurs that fall into particular frequency ranges. A diagnosis selection can be, for example, a pathology selection, an auscultatory finding selection, or the like. Auscultatory findings are acoustically perceived sounds and murmurs. The murmurs include, for example, such as systolic and diastolic murmurs, which can vary according to their timing (i.e., early, mid, late, holo, etc.), loudness that can be reflected in an assigned grade, and quality (i.e., soft, whispery, harsh, musical, etc). The sounds include, for example, the third sound (S3), fourth sound (S4), mid-systolic clicks, ejection sounds, opening snaps, pericardial knock and variations of the first (S1) and second (S2) sounds (i.e., splitting and difference in loudness, etc.), or the like. A particular pathology typically can have several auscultatory findings which result from the effect of the disease on the blood flow, pressure, velocity, and the like. An auscultatory finding can result from different diseases.

Some embodiments of the present disclosure are directed to an auscultation system that includes a digital filter capable of filtering the medical measurement signals with one or more diagnostic filters, where each diagnostic filter is operable to provide a band-pass filter to the medical measurement signals at one or more frequency ranges appropriate a particular diagnosis. The one or more frequency ranges appropriate to a particular diagnosis can be, for example, a frequency range covering frequency ranges of one or more auscultatory findings of a particular pathology, one or more frequency ranges corresponding to one or more auscultatory findings of a particular pathology, one or more frequency ranges corresponding to a particular auscultatory finding, or one or more frequency ranges corresponding to one or more pathologies associated with a particular auscultatory finding. A band-pass filter refers to a filter that attenuates frequencies outside the one or more frequency ranges. Such systems and methods may provide refined sound characteristics so a physician or other users can consider diagnosis possibilities that may not be practical using a typical stethoscope because such sound characteristics may be more difficult to appreciate without the filter. Such systems and methods may also allow some weak body acoustic signals to be captured so a pathology diagnosis becomes possible without additionally diagnosis tools, such as ultrasound or CT (Computed Tomography) Scan.

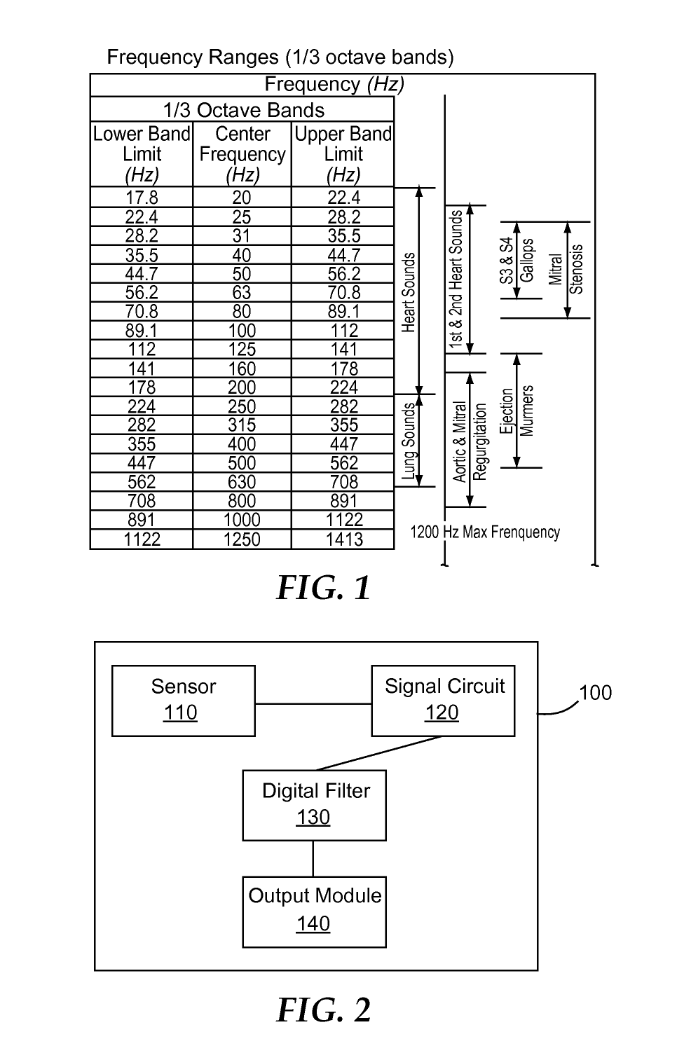

FIG. 1 illustrates human organ sounds range in terms of ⅓ octave frequency bands. For example, normal heart sounds are typically in the range of 20 Hz-200 Hz, while heart pathologies such as aortic and mitral regurgitation are typically in the range of 170 Hz-900 Hz. In order to provide a physician with a sound profile similar to the sound profile of a mechanical stethoscope, it may be desirable to have a similar sound profile in a frequency range of various body sounds. In some embodiments, this disclosure is directed to an auscultation system that has a frequency response that is close to a targeted frequency response, for example, a frequency response of a selected mechanical stethoscope, within a perceptually relevant criteria. The frequency response of the auscultation system refers to the overall frequency response of the system, which can include contributions from various components, for example, such as electronic circuit, digital filter, or the like. In certain implementations, this disclosure is directed to electrical stethoscope devices and systems using a digital filter that has a transfer function resulting in a frequency response of the system that is psychoacoustically equivalent to the frequency response of a particular mechanical stethoscope. Psychoacoustically equivalent, also referred to as perceptually equivalent, means that two sounds are generally psychologically and physiologically perceived as the same sound to a person.

Psychoacoustically equivalent can be determined by various measurement metrics. In some cases, a frequency response of an auscultation system is psychoacoustically equivalent to the frequency response of a particular mechanical stethoscope if the frequency response of the auscultation system is within a 3 dB deviation in the range of 20 Hz-1200 Hz to the frequency response of the particular mechanical stethoscope. In some other cases, a frequency response of an auscultation system is psychoacoustically equivalent to the frequency response of a particular mechanical stethoscope if the frequency response of the auscultation system is within a 2 dB deviation in the range of 20 Hz-2000 Hz to the frequency response of the particular mechanical stethoscope. In yet other cases, a frequency response of an auscultation system is psychoacoustically equivalent to the frequency response of a particular mechanical stethoscope if the frequency response of the auscultation system is within a 3 dB deviation from the frequency response of the particular mechanical stethoscope for each ⅓ octave frequency band in the range of 20 Hz-1200 Hz.

In some cases, a frequency response of an auscultation system is psychoacoustically equivalent to the frequency response of a particular mechanical stethoscope if the frequency response of the auscultation system is within a 1 dB deviation from the frequency response of the particular mechanical stethoscope for each ⅓ octave frequency band in the range of 20 Hz-1200 Hz. In some cases, a frequency response of an auscultation system is psychoacoustically equivalent to the frequency response of a particular mechanical stethoscope if the frequency response of the auscultation system is within a 1.5 dB deviation from the frequency response of the particular mechanical stethoscope for each ⅓ octave frequency band in the range of 20 Hz-1200 Hz. In some cases, a frequency response of an auscultation system is psychoacoustically equivalent to the frequency response of a particular mechanical stethoscope if the frequency response of the auscultation system is within a 2 dB deviation from the frequency response of the particular mechanical stethoscope for each ⅓ octave frequency band in the range of 20 Hz-1200 Hz.

In some cases, a frequency response of an auscultation system is psychoacoustically equivalent to the frequency response of a particular mechanical stethoscope if the frequency response of the auscultation system is within a 1 dB deviation from the frequency response of the particular mechanical stethoscope for each ⅓ octave frequency band in the range of 20 Hz-2000 Hz. In some cases, a frequency response of an auscultation system is psychoacoustically equivalent to the frequency response of a particular mechanical stethoscope if the frequency response of the auscultation system is within a 1.5 dB deviation from the frequency response of the particular mechanical stethoscope for each ⅓ octave frequency band in the range of 20 Hz-1200 Hz. In some cases, a frequency response of an auscultation system is psychoacoustically equivalent to the frequency response of a particular mechanical stethoscope if the frequency response of the auscultation system is within a 3 dB deviation from the frequency response of the particular mechanical stethoscope for each ⅓ octave frequency band in the range of 20 Hz-2000 Hz.

In some cases, a frequency response of an auscultation system is psychoacoustically equivalent to the frequency response of a particular mechanical stethoscope if the frequency response of the auscultation system is within a 1 dB deviation from the frequency response of the particular mechanical stethoscope for each ⅓ octave frequency band in the range of 30 Hz-700 Hz. In some cases, a frequency response of an auscultation system is psychoacoustically equivalent to the frequency response of a particular mechanical stethoscope if the frequency response of the auscultation system is within a 1.5 dB deviation from the frequency response of the particular mechanical stethoscope for each ⅓ octave frequency band in the range of 30 Hz-700 Hz. In some cases, a frequency response of an auscultation system is psychoacoustically equivalent to the frequency response of a particular mechanical stethoscope if the frequency response of the auscultation system is within a 2 dB deviation from the frequency response of the particular mechanical stethoscope for each ⅓ octave frequency band in the range of 30 Hz-700 Hz. In some cases, a frequency response of an auscultation system is psychoacoustically equivalent to the frequency response of a particular mechanical stethoscope if the frequency response of the auscultation system is within a 3 dB deviation from the frequency response of the particular mechanical stethoscope for each ⅓ octave frequency band in the range of 30 Hz-700 Hz.

In some cases, a frequency response of an auscultation system is psychoacoustically equivalent to the frequency response of a particular mechanical stethoscope if the frequency response of the auscultation system is within a 1 dB deviation from the frequency response of the particular mechanical stethoscope for each ⅓ octave frequency band in the range of 40 Hz-1000 Hz. In some cases, a frequency response of an auscultation system is psychoacoustically equivalent to the frequency response of a particular mechanical stethoscope if the frequency response of the auscultation system is within a 1.5 dB deviation from the frequency response of the particular mechanical stethoscope for each ⅓ octave frequency band in the range of 40 Hz-1000 Hz. In some cases, a frequency response of an auscultation system is psychoacoustically equivalent to the frequency response of a particular mechanical stethoscope if the frequency response of the auscultation system is within a 2 dB deviation from the frequency response of the particular mechanical stethoscope for each ⅓ octave frequency band in the range of 40 Hz-1000 Hz. In some cases, a frequency response of an auscultation system is psychoacoustically equivalent to the frequency response of a particular mechanical stethoscope if the frequency response of the auscultation system is within a 3 dB deviation from the frequency response of the particular mechanical stethoscope for each ⅓ octave frequency band in the range of 40 Hz-1000 Hz.

FIG. 2 illustrates a block diagram of an exemplary embodiment of an auscultation system 100. An auscultation system, also referred to as an electrical stethoscope, can be any electronic system functioning as a stethoscope. For example, an auscultation system can include physically connected sensor(s), a signal processing circuit, a digit filter, and an output component. As another example, an auscultation system can include both local components and remote components to generate medical measurement signals, process the medical measurement signals, filter the processed medical measurement signals, and output the filtered signals. As used herein, local components are components electronically coupled to the sensors. A digital filter, as used herein, can be a digital circuit providing desired filtering functions, a processor unit that is programmed with desired filtering functions, or a combination of a digital circuit and a processor unit to realize such desired filtering functions. The processor unit includes but is not limited to one or more digital signal processors (DSP), microcontrollers, microprocessors, computers, handheld computers (e.g., tablets), cellular phones.

In some embodiments, the auscultation system 100 can include a sensor 110, an optional signal circuit 120, a digital filter 130, and an output module 140. The components of the auscultation system 100 can be hosted in a single housing or more than one housings with wired or wireless connections. The sensor 110 is configured to sense sounds produced by matter of biological origin, such as sounds produced by the heart, lungs, vocal cords, or other organs or tissues of the body, and generate medical measurement signals. In some embodiments, the sensor 110 of the electronic stethoscope is configured to modulate or generate a medical measurement signal in response to deformation of the transducer. Suitable transducers are those that incorporate piezoelectric material (organic and/or inorganic piezoelectric material) such as piezoelectric film, piezoresistive material, strain gauges, capacitive or inductive elements, a linear variable differential transformer, and other materials or elements that modulate or generate an electrical signal in response to deformation. The sensor 110 may be planar or non-planar, such as in the case of a curved or corrugated configuration. Suitable piezo materials may include polymer films, polymer foams, ceramic, composite materials or combinations thereof. The sensor 110 may incorporate arrays of transducers of the same or different transducer type and/or different transducer materials, all of which may be connected in series, individually, or in a multi-layered structure. Suitable transducers that incorporate plural sensing elements having differing characteristics and/or sensors with tailorable sensing characteristics are disclosed in commonly owned U.S. Patent Application Publication Nos. 2007/0113649 and 2007/0113654, each of which is incorporated herein by reference in its entirety.

The sensor 110 may be implemented to generate medical measurement signals using technologies other than those that employ electromagnetic energy or piezo materials. For example, the sound to be transduced may move a cantilever that has a highly reflective surface, and a laser or optical beam of light shining on this surface may be modulated. The intensity or other property of the modulated light may be received by a photodetector that outputs an electrical signal for analysis. As a further example, one or more accelerometers may be employed to sense sound signals and produce medical measurement signals corresponding to the sound signals.

The signal circuit 120 is configured to receive the medical measurement signals generated by the sensor 110. In some embodiments, the medical measurement signals are analog signals and the signal circuit 120 is configured to convert the medical measurement signals to digital medical measurement signals. In some implementations, the signal circuit 120 is further configured to provide analog amplification and/or analog filtering to the medical measurement signals before the analog-to-digital conversion. The signal circuit 120 can precondition medical measurement signals based on the desired characteristics of the medical measurement signals.

The digital filter 130 is configured to provide digital filtering to the processed medical measurement signals and generate filtered signals. In some embodiments, the digital filter 130 is configured to provide one or more filters, where each filter is operable to provide a transfer function resulting in a frequency response similar to that of a mechanical stethoscope. A frequency response of the auscultation system 100 depends on the design of the signal circuitry and the filter used in the digital filter 130. In some embodiments, the digital filter 130 is configured to provide one or more filters, where each filter is operable to provide a transfer function resulting in a frequency response of the auscultation system 100 that is close to the frequency response of a selected mechanical stethoscope within a predetermined threshold. In some cases, the predetermined threshold can be a threshold that is perceptually relevant to users. For example, the predetermined threshold can be a 2 dB deviation for a frequency range of 10 Hz-3000 Hz. In a particular embodiment, the digital filter 130 is configured to provide one or more filters, where each filter is operable to provide a transfer function resulting in a frequency response of the auscultation system 100 that is within a 3 dB deviation for each ⅓ octave frequency band in the range of 20 Hz-1200 Hz to the frequency response of a mechanical stethoscope. In another embodiment, the digital filter 130 is configured to provide one or more filters, where each filter is operable to provide a transfer function resulting in a frequency response of the auscultation system 100 that is within a 3 dB deviation in the range of 20 Hz-1200 Hz to the frequency response of a mechanical stethoscope.

In some embodiments, the output module 140 can include a signal presentation device and/or a communication device. In some implementations, the output module 140 can include a headset. The headset typically includes binaural tubes and ear tips. In some cases, the filtered signals can be amplified before the signals are sent to the headset. In some other implementation, the output module 140 can include a communication device, for example, a wired or wireless transmitter, to transmit the filtered signals.

In some embodiments, the auscultation system 100 can be a stethoscope system suitable for telemedicine applications. In such configurations, one or more components of the signal circuit 120, the digital filter 130, and the output module 140 can locate remotely from the sensor 110. In some implementations, the output module 140 can locate remotely from the sensor 110, where the sensor 110, the signal circuit 120, and the digital filter 130 can be local components. In such implementations, the output module 140 can include a receiver and components for presenting the filtered signals. In an exemplary embodiment, the output module 140 can include a headset and a wireless receiver while the auscultation system 100 can include a wireless transmitter coupled to the digital filter 130, where the transmitter is configured to transmit the filtered signals. In one embodiment, the auscultation system 100 can include a transmitter coupled to the signal circuit 120, where the transmitter is configured to send the processed medical measurement output from the signal circuit 120, and a receiver coupled to the digital filter 130 located remotely from the sensor, where the receiver is configured to receive the processed medical measurement signals and pass the signals to the digital filter for further filtering.

In some implementations, the sensor 110, the signal circuit 120, and the digital filter 130 can be hosted in a single housing. In some other implementations, the auscultation system 100 may include a local part including the sensor 110 and a remote part including the output module 140 such that the auscultation system 100 is suitable to be used in telemedicine applications. In some implementations, the remote part includes both the digital filter 130 and the output module 140. In some other implementations, the remote part can include part of the digital filter 130 and the output module 140. In an exemplary embodiment, the digital filter 130 coupled with a wireless receiver is configured to receive the processed medical measurement signals sent from the signal circuit 120 and the filtered signals are presented by the output module 140. As another example, the remote part includes the output module 140 where the output module 140 may receive the filtered signals by a wireless means and deliver the filtered signals to an audio device.

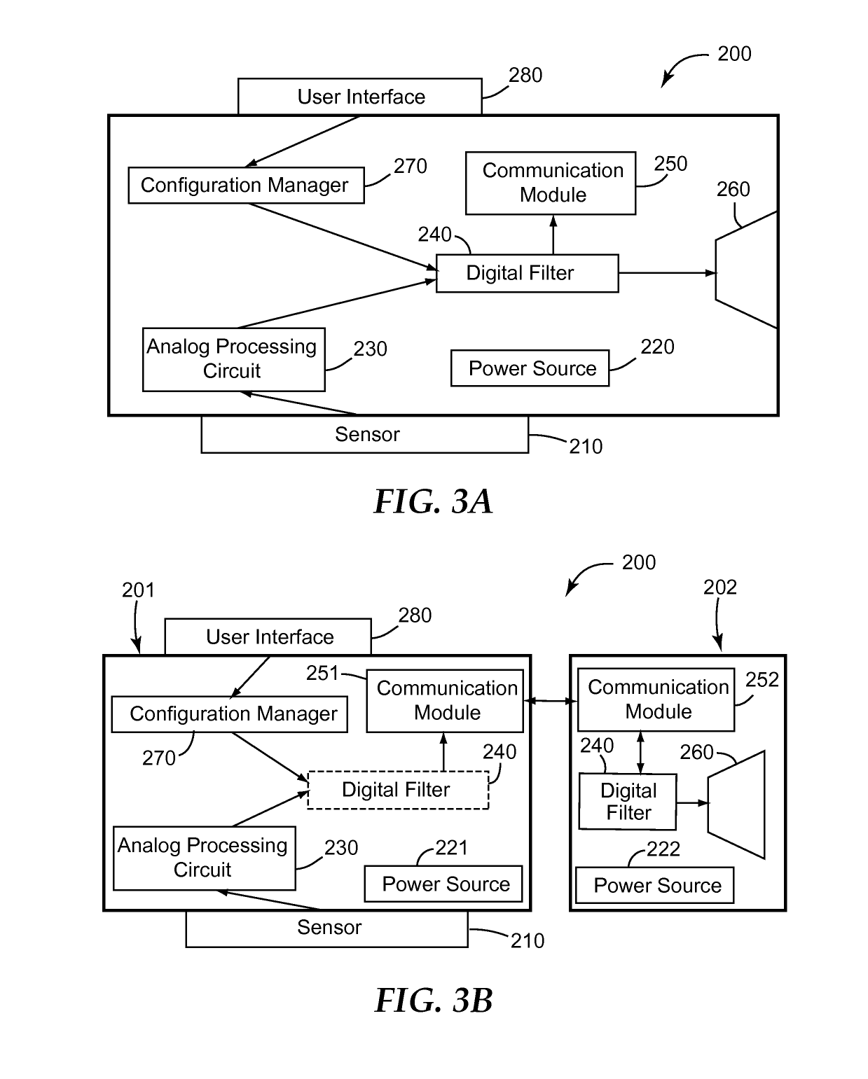

FIG. 3A illustrates an exemplary system diagram of an auscultation system 200. In the depicted embodiments, the auscultation system 200 includes a sensor 210, a power source 220, an analog processing circuit 230, a digital filter 240, an output device 260, a configuration manager 270, and a user interface 280. The sensor 210 is configured to generate medical measurement signals from sounds detected, for example, from a human body. The power source 220 may be designed to provide the requisite power for a particular stethoscope configuration. As the configuration of the stethoscope is changed over time, for example, the power source may be changed to accommodate the power supply requirements of each configuration change to the stethoscope. The power source 220 may differ in terms of chemistry, form factor, rechargeability, and capacity, for example. The power source 220 may be fabricated to provide a single power source or multiple power sources. For example, a primary power source may be implemented as the main source of power for the electronics of the stethoscope. A secondary power source may be a storage capacitor or battery smaller than the primary power source, and used for powering transducers or circuitry during sleep mode or for detecting conditions for transitioning the stethoscope from sleep mode to operational status. As another example, the power source 220 may include a first power source to supply power to a local part of the stethoscope and a second power source to supply power to a remote part of the stethoscope. In some implementations, the power source 220 can include power management circuitry such as that described in U.S. Patent Application Publication No. 2008/0232604, entitled “Power Management for Medical Sensing Devices Employing Multiple Sensor Signal Feature Detection,” which is incorporated herein by reference in its entirety.

In some embodiments, the auscultation system 200 can include a user interface 280. The user interface 280 may include a number of mode and/or status indicators and mode and/or control switches. The switches may include volume or gain control switches and mode selection switches, for example. In a particular embodiment, the mode selection switches can provide a number of stethoscope sound profiles for user's selection. The indicators may provide an indication of a selected filter mode, or other information, such as battery and communication link status. In some implementations, the user interface 280 can include a display, for example, a touch sensitive display. In some cases, the display is capable of providing a list of filters or sound profiles. A user can select a filter from the list of filters, by pressing a button or a position on the display, for example, depending on the type of display and input methods.

In some embodiments, the configuration manager 270 can take the input from the user interface 280 and change the configuration of the auscultation system 200 accordingly. For example, the configuration manager 270 can take a selected filter mode to change the filter configuration used in the digital filter 240. As another example, the configuration manager 270 may change one or more amplification factors based on input received from the user interface 280, for example, from gain control switches. The configuration manager can be implemented on a programmed processing unit or by circuitry. The analog processing circuit 230 can provide preliminary filtering and amplification to signals generated by the sensor 210 and output processed signals to the digital filter 240. The digital filter 240 can provide filtering to the processed signals and output the filtered signals to the output device 260 such that the auscultation system 200 may have a sound profile similar to a mechanical stethoscope. In some embodiments, the digital filter 240 can include a number of filter selections, where each filter selection simulates a mechanical stethoscope respectively, for example, LITTMANN Master Cardiology, LITTMANN Cardiology III, LITTMANN Classic II, LITTMANN Cardiology S.T.C., or the like. In such implementations, the selection of the desired mechanical stethoscope simulation may be selected via the user interface 280. In a particular embodiment for simulating the sound profile of a mechanical stethoscope in a frequency range sufficiently broad, the digital filter 240 can provide a transfer function that results in a frequency response of the auscultation system 200 within a 3 dB deviation from the frequency response of the mechanical stethoscope in the frequency range of 20 Hz-1200 Hz. In another embodiment, the digital filter 240 can provide a transfer function that results in a frequency response of the auscultation system 200 within a 2 dB deviation from the frequency response of the mechanical stethoscope in the frequency range of 20 Hz-1200 Hz.

In some embodiments, the auscultation system 200 can optionally include a communication module 250 to communicate filtered signals by wired and/or wireless connection. The wired connection may be via interfaces of a variety of protocol, for example, Universal Serial Bus (USB), Mini USB, FireWire™ (IEEE 1394 Interface), internet, or other communication protocol. In addition, the communication module 250 may be configured to connect to a docking station that interfaces with a computing device. When connected, recharging power may also be delivered to the auscultation system 200 via a wired connection port. The attachment of the auscultation system 200 to the cable or docking station can trigger the automatic launch of control/application software on the computing device and/or allow sound or data files stored on the auscultation system 200 to upload or synchronize into the computing device. In some implementations, the computing device can be the output device 260.

The communication module 250 can support a wireless connection with any short-range or long range wireless interfaces. The short-range communication interfaces may be, for example, interfaces conforming to a known communications standard, such as Bluetooth standard, IEEE 802 standards (e.g., IEEE 802.11), a ZigBee or similar specification, such as those based on the IEEE 802.15.4 standard, or other public or proprietary wireless protocol. The long-range communication interfaces may be, for example, cellular network interfaces, satellite communication interfaces, or the like. The communication module 250 may utilize a secured communication link.

In some embodiments, the digital filter 240 or a separate processing unit (not shown in FIG. 3) may process the filtered signals to provide various output data, such as a visual, graphical and/or audible representation of the information (e.g., heart rate indication, S1-S4 heart sounds), and/or diagnostic information regarding anomalous cardiac, lung, or other organ function (e.g., phonocardiogram, frequency spectrogram, cardiac murmurs such as those resulting from valve regurgitation or stenosis, breathing disorders such pneumonia or pulmonary edema), or other organ pathology. In some implementations, the separate processing unit can be part of the output device 260.

In some embodiments, the auscultation system 200 may include a local part 201 and a remote part 202, as illustrated in FIG. 3B. In one embodiment, the local part 201 can include a sensor 210, an analog processing circuit 230, a first power source 221, a user interface 280, a configuration manager 270, and a first communication module 251. The remote part 202 can include a digital filter 240, a second power source 222, a second communication module 252, and an output device 260. In another embodiment, the digital filter 240 can be disposed in the local part 201. In yet another embodiment, the digital filter 240 can be implemented by a two-part circuitry or processing unit, one part of the digital filter 240 located in the local part 201 and the other part of the digital filter 240 located in the remote part 202. In some cases, the first communication module 251 and the second communication module 252 can include wireless transceivers capable of transmitting and receiving data via a wireless connection. In some other cases, the first communication module 251 and the second communication module 252 can include wired connection ports capable of transmitting and receiving data via a wired connection.

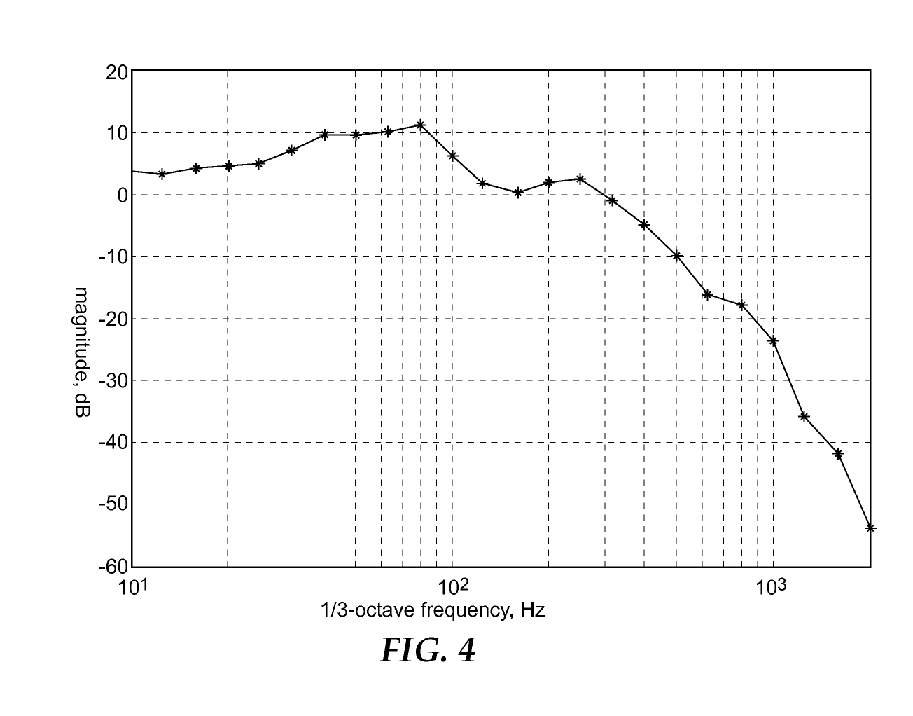

FIG. 4 illustrates a frequency response of an exemplary mechanical stethoscope, in particular, the ⅓ octave frequency response curves for the LITTMANN Master Cardiology Stethoscope. To simulate a transfer function of a mechanical stethoscope, a filter-fit method can be used, for example, to implement a multi-stage Infinite Impulse Response (IIR) filter.

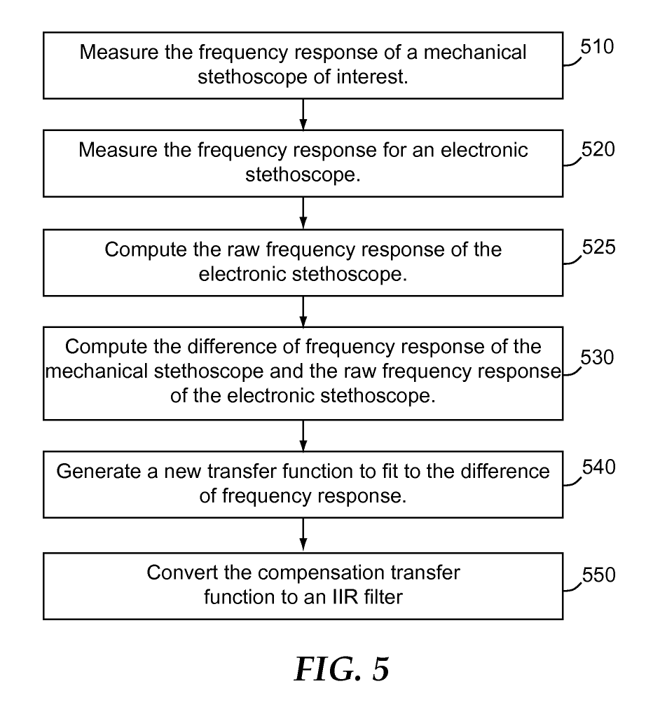

FIG. 5 illustrates an exemplary logical flowchart of an embodiment of designing a stethoscope simulation filter. Initially, the frequency response of a mechanical stethoscope of interest is measured, which can be denoted as H_{M}(z) (step 510). The frequency response of the electronic stethoscope is also measured, which can be denoted as H_{E}(z) (step 520). Next, the raw frequency response of the electronic stethoscope can be computed, which can be denoted as H_{R}(z) (step 525). The raw frequency response of the electronic stethoscope refers to the frequency response generated by the underlying circuit design and other effects not including the digital filter in use. The raw frequency response can be computed by subtracting the frequency response of the electronic stethoscope from the frequency response of the digital filter in use (i.e., a diaphragm filter, a bell filter, etc.), which can be denoted as H_{R}(z)=H_{E}(z)−H_{I}(z), where H_{I}(z) represents the frequency response of the digital filter in use. The difference between the raw frequency response of the electronic stethoscope and the frequency response of the mechanical stethoscope of interest is computed, which can be denoted as H_{D}(z)=H_{M}(z)−H_{R}(z), (step 530). Generate a new transfer function, which can be denoted as H_{N}(z), to fit to H_{D}(z) using a curve fitting technique (step 540). In some implementations, the curve fitting technique can be implemented using a MATLAB script. In a particular embodiment, the curve fitting of complex transfer functions can include fitting for both magnitude and phase. The new transfer function, H_{N}(z), can be converted to an IIR filter (step 550). The electronic stethoscope can implement a stethoscope simulation filter using the designed IIR filter. In some cases, the frequency response of anelectronic stethoscope using the desired IIR filter can be measured to verify that this filter adequately matches the frequency response of the mechanical stethoscope. In some cases, this step can include verifications on both magnitude and phase response.

In an exemplary embodiment, frequency response of a stethoscope can be measured using a head acoustic simulator. The ear tips for the stethoscope under test are inserted into the left and right ears of the head acoustic simulator. During the frequency response measurement, a pink noise recording is played in an enclosed loudspeaker with an opening above. The opening is filled by a gel or foam pad supported by a screen. The stethoscope chestpiece is placed on the pad and is loaded with a 50 gram weight to simulate light pressure or 450 gram for firm pressure.

All signal inputs and output are linked to a computing device using the input/output module of a signal analyzer. A power amplifier may be needed to drive the loudspeaker. Signal outputs from the head simulator microphones are fed into the I/O module inputs. A reference microphone placed directly above the loudspeaker but below the gel pad can be used. In some cases, the signal from the reference microphone can be the denominator of the transfer function calculation for frequency response and the left or right ear microphone signal is the numerator.

Signal analysis software can be used to integrate microphone signals over a 5 to 10 second interval and compute the complex frequency response using Fast Fourier analysis. Digitally sampled signals are analyzed in the discrete time domain (z) using digital signal processing.

In an exemplary embodiment, after the new transfer function, H_{N}(z), is determined, a cascade of 2 second order IIR filters may be used to implement a tenth order IIR compensation filter to realize the transfer function.

H_{N}(z)=b₀H₁(z)H₂(z)H₃(z)H₄(z)H₅(z)

H_{N}(z) is decomposed into to the product of 5 digital biquad filters in a second order recursive linear form. For the second order Direct Form 5 realization, the coefficients each second order section have the form.

$${H}_{i} ( z ) = \frac{{b}_{i 0} + {b}_{i 1} {z}^{- 1} + {b}_{12} {z}^{- 2}}{1 + {a}_{i 1} {z}^{- 1} + {a}_{i 2} {z}^{- 2}} ,$$

where the variable i is 1 through 5.
The difference equations for the biquad Direct Form 2 realization can be written as

yᵢ(n)=bᵢ₀w(n)+bᵢ₁w(n−1)+bᵢ₂w(n−2)

where

wᵢ(n)=x(n)−aᵢ₁w(n−1)−aᵢ₂w(n−2).

Table 1 illustrates a set of filter coefficients for LITTMANN Model 3200 to simulate a LITTMANN Master Cardiology Stethoscope.

TABLE 1

| Biquad | | | | | | |
|---|---|---|---|---|---|---|
| Filter | b0 | b1 | b2 | a0 | a1 | a2 |
| 1 | 0.433602699 | −0.756275377 | 0.379785360 | 1 | −1.762362171 | 0.933505576 |
| 2 | 0.619514557 | −1.220059694 | 0.607905048 | 1 | −1.943502411 | 0.946009495 |
| 3 | 0.239057938 | −0.791645048 | 0.535155242 | 1 | −1.535553441 | 0.870959755 |
| 4 | 0.220192745 | −0.354242182 | 0.200238840 | 1 | −1.911197115 | 0.914975882 |
| 5 | −0.134066468 | −0.134066468 | −0.268132936 | 1 | 0.009403885 | −0.990473499 |

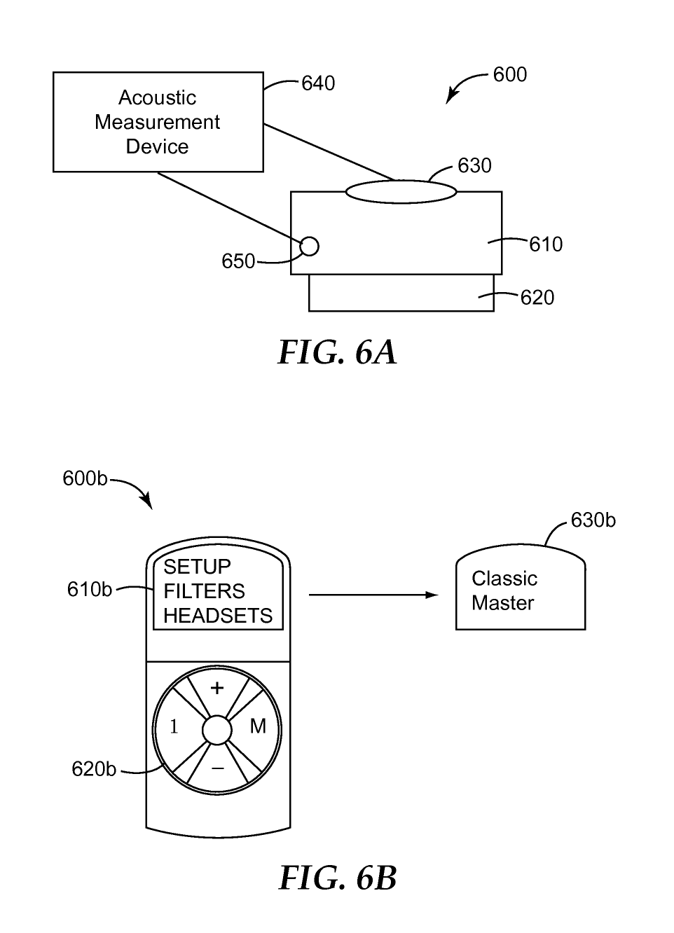

A variety of test methods can be used to test the performance of an electronic stethoscope to ensure that the simulation to the mechanical stethoscope is within the desired deviation in frequency response. For example, an air coupling test method can be used. FIG. 6A illustrates a schematic view of an acoustic test system 600 to test frequency response of a stethoscope. The acoustic test system 600 can include an acoustic medium 610, an acoustic source 620, a stethoscope 630, and an acoustic measurement device 640. The acoustic medium 610 can provide a cavity coupling between the acoustic source 620 and the stethoscope 630. The acoustic medium 610 can comprise one or more coupling medium, for example, such as air, liquid, gel, foam, or the like. The acoustic medium 610 can have the shape of, for example, cylinder, cube, or the like. In some cases, the acoustic medium 610 can be sealed. The acoustic medium 610 can also provide support to the placement of the stethoscope 630. The sensor of the stethoscope 630 typically faces the cavity of the acoustic medium 610 to detect sound signals generated by the acoustic source 620. The acoustic source 620 can be, for example, a voice coil, a loudspeaker, or the like. The acoustic measurement device 640 is capable of detecting the acoustic signals. The acoustic measurement device 640 can be, for example, a Brüel & Kjær PULSE Analyzer, or a National Instrument acoustic testing system. In some implementations, a microphone 650 can be optionally included and placed inside the cavity of the acoustic medium 610 to provide reference signals. In such implementations, the frequency response of the stethoscope 630 can be indicated as the ratio of the output signals of the stethoscope verse the reference signals generated from the microphone 650 at each frequency band. In an exemplary embodiment, the acoustic test system 600 for measuring the frequency response of LITTMANN Model 3200 includes an acoustic medium providing air cavity, a loudspeaker as the acoustic source, and a Brüel & Kjær PULSE Analyzer as the acoustic measurement device.

FIG. 6B illustrates an exemplary user interface 600b for filter selection. In some embodiments, the digital filter is capable of filtering the processed medical signals with a plurality of filters and the user interface 600b is configured to allow a user to select a filter from the plurality of filters. After the filter selection is made, the digital filter is configured to filter the processed medical signals with the selected filter.

The user interface 600b may include a graphical user interface 610b and a control section 620b. In some implementations, the graphical user interface 610b can be a display to present, for example, configuration information, status information, and measurement data. Additionally, the graphical user interface 610b can be a touch sensitive device that accepts user input on the screen. The control section 620b can include a number of switches and buttons that allow users to change configuration of the stethoscope. As an example, the display content of the graphical user interface 610b can be a list of digital filters, as illustrated in a graphical user interface 630b. In an exemplary embodiment, the list of digital filters can include the filters corresponding to transfer functions simulating to transfer functions of mechanical stethoscopes. Depending on the implementation, a user may select a digital filter from the list by buttons or switches selection, pressing on a touch sensitive device, or other mechanical or electronic means.

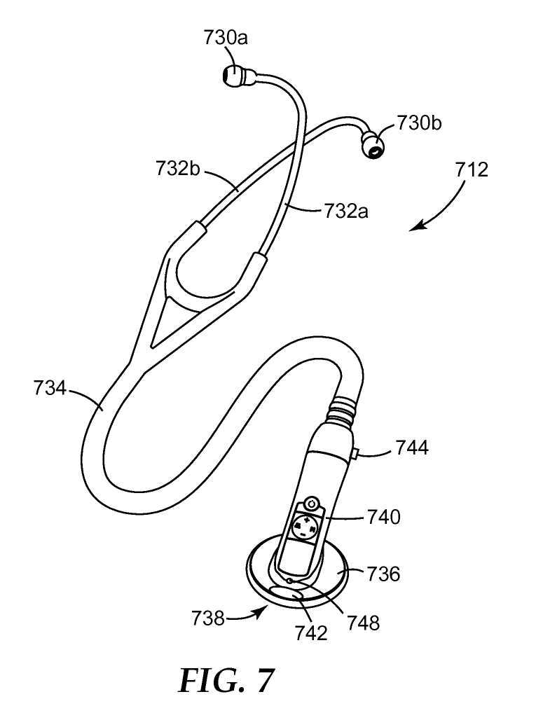

FIG. 7 illustrates is a perspective view of one embodiment of an electronic stethoscope 712. In one embodiment, the electronic stethoscope 712 includes ear tips 730a, 730b, ear tubes 732a, 732b, and a main tube 734. The main tube 734 is coupled to a main housing or chestpiece 736, which supports one or more sensors. The signal processing circuitry of the electronic stethoscope 712, including a digital filter and other optional circuitry, can be configured to provide transfer functions simulating to transfer functions of mechanical stethoscopes. The signal processing circuitry is further configured to convert the signals generated by the sensor to acoustic signals for transmission through the ear tubes 732a, 732b to reproduce body sounds through the ear tips 730a, 730b. In some embodiments, the reproduced body sounds have a sound profile simulated to a mechanical stethoscope.

The electronic stethoscope 712 can include a user interface 740. The user interface 740 may include one or more switches, electronic displays, indicators, and other input and output devices. The electronic stethoscope 712 can also include an integrated communication system that provides wired and/or wireless communication. In some embodiments, an antenna (not shown) for the communication system is integrated into the main housing 736. In order to improve the communication link with the electronic stethoscope 712, an aperture 742 may be formed in the metal main housing 736 and covered with a more electromagnetically transparent material. For example, the aperture 742 can be covered with a polymeric member. A flashing light source (e.g., LED) may be mounted in the aperture to indicate that the wireless connection is active, and to remind the user of the electronic stethoscope 712 to not cover the aperture 742. A return signal strength indicator may be included on the user interface 740 to provide the strength of the communication link to the user while a connection with the computer is established. In some embodiments, a small parabolic reflector is placed under the antenna to reflect signals transmitted from the antenna normally lost into the tissue of the patient, and to concentrate signals received from the computer captured by the antenna. In an alternative embodiment, the antenna is mounted in one of the ear tubes 732a, 732b or the main tube 734 to locate the antenna higher and improve the line-of-sight with another wireless transmitter. The antenna may include multiple branches that are mountable on both sides of the ear tubes 732a, 732b to allow unobstructed signal communication under varying body orientations.

In one embodiment, the electronic stethoscope 712 may also include a wired connection port 744 to allow a wired connection between the electronic stethoscope 712 and an external device (e.g., personal computer, personal digital assistant (PDA), cell phone, netbook, tablet computer, etc.). A conductor (electrical or optical) may be connected between the wired connection port 744 of the electronic stethoscope 712 and an appropriate connector on the external device. The wired connection port 744 of the electronic stethoscope 712, and any necessary interface circuitry, may be configured to communicate information in accordance with a variety of protocols, such as FireWire™ (IEEE 1394), USB, Mini USB, or other communications protocol. In addition, the connection port 744 may be configured to connect to a docking station that interfaces with the electronic stethoscope 712 and the external device. The attachment of the electronic stethoscope 712 to the cable or docking station can trigger the automatic launch of control/application software on the external device and/or allow sound or data files stored on the electronic stethoscope 712 to upload or synchronize into the external device. When connected, recharging power may also be delivered to the electronic stethoscope 712 via the wired connection port 744.

An acoustic transducer or microphone 748 may also be integrated into the top side (i.e., the side facing away from the sensor) of the main housing 736. The microphone 748 may be used to receive ambient sounds from the area surrounding the microphone 748. For example, the microphone 748 may be used, in addition to or in lieu of sensor, to pick up voice signals from the user of the electronic stethoscope 712.

In some embodiments, the electronic stethoscope 712 can include an integrated electronic storage medium that allows a user to store voice tracks, body sounds, or other recordings in the electronic stethoscope 712 for later review. The electronic storage medium may further include voice recognition data to identify the user or owner of the stethoscope and speech recognition data to identify voice commands so that certain settings (e.g., power, volume) of the electronic stethoscope 712 may be modified in response to voice commands. Speech recognition voice commands may also be used to transfer voice tracks, body sounds, or other recordings or files to a patient medical record database. In some embodiments, the electronic stethoscope is configured to transcribe the content of voice signals into records or other data files (e.g., patient medical records), as described, for example, in U.S. Pat. No. 7,444,285. The voice tracks may also be stored with sound tracks relating to sensed body sounds such that the body sounds and voice tracks can be played back simultaneously through the ear tips 730a, 730b. In some embodiments, the user interface 740 allows the user to scroll through the body sounds and voice tracks stored in the electronic storage medium for selection and playback. The microphone 748 may also be employed for active ambient noise reduction to remove unwanted surrounding environmental noise from the recorded body and voice signal.

FIG. 8 illustrates an exemplary embodiment of an auscultation system 800 adapted for telemedicine applications. The auscultation system 800 includes a wireless chestpiece 820 and a wireless headset 822. The wireless chestpiece 820 is configured substantially similarly to the chestpiece 736 described above with regard to FIG. 7. The wireless chestpiece 820 can be configured to connect with other components of a telemedicine system via a secure network connection. Components that may be disposed in the chestpiece 820 include a power source, signal processing circuitry, and a communications interface. A sensor 824 is supported at one end of the wireless chestpiece 820, and an antenna 826 is mounted at an end of the wireless chestpiece 820 opposite the sensor 824. The sensor 824 may have properties and configurations similar to those described above with regard to sensor. In the embodiment shown, the antenna 826 is configured to swivel or rotate about pivot 828 to allow the antenna 826 to be positioned for maximum signal coupling during use. The antenna 826 can also be positioned to minimize clearance during storage. In some embodiments, the antenna 826 is a high performance antenna for large signal range (e.g., greater than 100 m), thereby maximizing the mobility of the wireless chestpiece 820.

The wireless headset 822 is configured to receive signals from the wireless chestpiece 820 via a wireless connection. In a preferred embodiment, the wireless headset 822 and the wireless chestpiece 820 can be configured to communicate via a secure connection. In some embodiments, the chestpiece 820 and headset 822 are paired with each other via a Bluetooth connection. The wireless headset 822 may also be configured to communicate with other components of a telemedicine system. The wireless headset 822 may have various configurations, including over-the-ear and in-ear designs. In the embodiment shown, the wireless headset 822 includes ear tips 830a, 830b for in-ear use. In some embodiments, the ear tips 830a, 830b are substantially the same as ear tips 730a, 730b in FIG. 7 to provide consistent sound quality to the user between the electronic stethoscope 712 and the auscultation system 800.

In some embodiments, the signal processing circuitry of the wireless chestpiece 820 can have the same components as the circuitry of the electronic stethoscope 712 as illustrated in FIG. 7. In some embodiments, the output body sound signals from the wireless chestpiece 820 presents a sound profile deliberately similar to a sound profile of a mechanical stethoscope. In a particular embodiment, the output signals from the wireless chestpiece 820 have frequency response within a 3 dB deviation from the frequency response of the mechanical stethoscope within the frequency range of 20 Hz-1200 Hz In some other embodiments, the wireless headset 822 can also include a signal processing circuitry, where the combined functions of the signal processing circuitry of the wireless chestpiece 820 and the signal processing circuitry of the wireless headset 822 is equivalent to the signal processing circuitry of the electronic stethoscope 712 as illustrated in FIG. 7. For example, an analog signal processing circuitry and an analog-to-digital converter are disposed in the wireless chestpiece 820 and a digital filter is disposed in the wireless headset 822.

The wireless headset 822 may also include a microphone 832 for receiving voice signals from the user. The microphone 832 is coupled to an adjustable support 834 that allows the microphone 832 to be repositioned relative to the user. The signals received by the microphone 832 may be superimposed over the body sounds sensed by the chestpiece 820 and sent over the wireless connection, as described above.

The wireless headset 822 can be a variety of headsets, for example, such as 3M ComTac headset, Bose A20 headset, or the like. Because headsets typically have their own characteristic frequency response, it can be desirable to compensate for this response to achieve the desired stethoscope frequency response. Referring back to FIG. 1, the auscultation system can include a digital filter that is capable of filtering the medical measurement signals with one or more headset filters, where each headset filter operable to provide a transfer function to compensate for the characteristic frequency response of a particular headset.

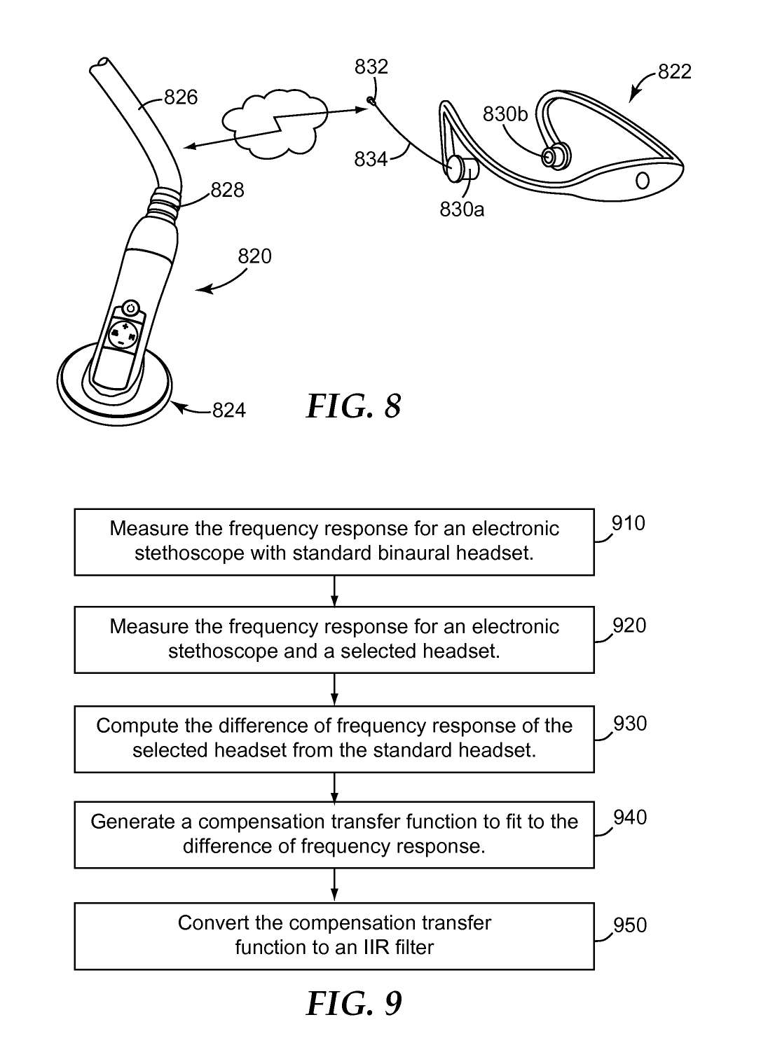

FIG. 9 illustrates an exemplary logical flowchart of an embodiment of designing a headset filter. Initially, the frequency response of an electronic stethoscope with a standard binaural headset is measured, which can be denoted as H_{E}(z) (step 910). The frequency response of the electronic stethoscope with a selected headset is also measured, which can be denoted as H_{H}(z) (step 920). The difference between the frequency response of the electronic stethoscope with a standard headset and with a selected headset is computed, which can be denoted as H_{D}(z)=H_{E}(z)−H_{H}(z), (step 930). Generate a compensation transfer function, H_{C}(z), to fit to H_{D}(z) using a curve fitting technique (step 940). In some cases, the curve fitting technique can be implemented with a MATLAB script. In a particular embodiment, the curve fitting of a complex transfer function can include fitting for both magnitude and phase. The compensation transfer function, H_{C}(z), is converted to an IIR filter (step 950). The electronic stethoscope can implement a headset filter using the designed IIR filter.

In an exemplary embodiment, after the compensation transfer function, H_{C}(z), is determined, a cascade of 2 second order IIR filters may be used to implement a tenth order IIR compensation filter to realize the transfer function.

H_{C}(z)=b₀H₁(z)H₂(z)H₃(z)H₄(z)H₅(z)

H_{C}(z) is decomposed into to the product of 5digital biquad filters in a second order recursive linear form. For the second order Direct Form 2 realization, the coefficients each second order section have the form.

$${H}_{i} ( z ) = \frac{{b}_{i 0} + {b}_{i 1} {z}^{- 1} + {b}_{12} {z}^{- 2}}{1 + {a}_{i 1} {z}^{- 1} + {a}_{i 2} {z}^{- 2}} ,$$

where i is 1 through 5.
The difference equations for the biquad Direct Form 2 realization can be written as

yᵢ(n)=bᵢ₀w(n)+bᵢ₁w(n−1)+bᵢ₂w(n−2)

where

wᵢ(n)=x(n)−aᵢ₁w(n−1)−aᵢ₂w(n−2).

Table 2 illustrates a set of filter coefficients of an exemplary headset filter.

TABLE 2

| Biquad Filter | b0 | b1 | b2 | a0 | a1 | a2 |
|---|---|---|---|---|---|---|
| 1 | 0.3804 | −0.6669 | 0.3142 | 1 | −1.8865 | 0.9375 |
| 2 | 0.6133 | −0.4918 | 0.3440 | 1 | −0.7376 | 0.9207 |
| 3 | 0.2040 | −0.4364 | 0.5411 | 1 | −1.7147 | 0.8910 |
| 4 | 0.4328 | 0.0466 | 1.2015 | 1 | −1.4391 | 0.8541 |
| 5 | −2.6411 | 4.5066 | −0.8109 | 1 | −0.0895 | 0.8507 |

The auscultation system can include a user interface that is configured to allow a user to select a headset filter from one or more headset filters. The digital filter is configured to filter medical measurement signals with the selected headset filter.

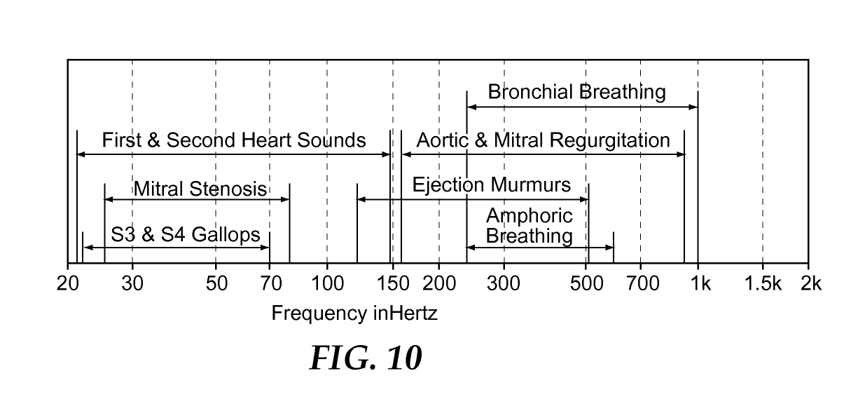

FIG. 10 illustrates some exemplary pathologies. A pathology diagnosis is typically contained within one or more corresponding frequency ranges. For example, an ejection murmur is typically in the frequency range of 120 Hz-500 Hz. In some cases, the frequency range can be changed with the severity of a disease. In some other cases, a particular pathology can associate with one or more auscultatory findings. Mitral stenosis can include the auscultatory findings of, for example, mid-diastolic murmur, an unusually loud first heart sound, or other sounds depending on the severity. In some embodiments, the digital filter is configured to provide one or more diagnostic filters, where each diagnostic filter is operable to provide a band-pass filter corresponding to the medical measurement signals at one or more frequency range appropriate to a particular diagnosis. The band-pass filter can pass signals within the one or more frequency ranges and attenuate signals outside the one or more frequency ranges. In some cases, the diagnostic filter can filter medical measurement signals with frequency ranges corresponding to more than one pathology diagnosis. In some other cases, the diagnostic filter can filter medical measurement signals with frequency ranges corresponding to one or more auscultatory findings. In yet other cases, the diagnostic filter can filter medical measurement signals with frequency ranges corresponding to one or more particular auscultatory findings related to a pathology diagnosis. In some other cases, the diagnostic filter can filter medical measurement signals with frequency ranges corresponding to a particular auscultatory finding related to one or more pathology diagnosis.

In some embodiments, the auscultation system can include a user interface that is configured to allow a user to select a diagnostic filter from the one or more diagnostic filters. In some cases, the user interface can provide a list of pathology or a list of auscultatory findings for a user to select. In some implementations, after a pathology selection is made, the user interface can also allow a user to specify the severity level, one or more particular auscultatory findings, or other characteristics of the selected pathology. For example, the user can select a severity level among severity levels of trace, mild, moderate, moderate severe, and severe. In some other implementations, after an auscultatory finding is selected, the user interface can also allow a user to select one or more particular pathologies associated with the selected auscultatory finding. As used herein, a particular diagnosis refers to one or more selected pathologies, one or more specified characteristic of the selected pathologies, one or more selected auscultatory findings, one or more specified pathologies associated with the selected auscultatory findings. For example, a particular diagnosis can be mitral stenosis, mild mitral stenosis, or mid-diastolic murmur of mitral stenosis. In some implementations, a diagnostic filter can be a band-pass filter to filter signal at one or more frequency ranges appropriate to a particular diagnosis. The digital filter is then configured to filter the medical measurement signals with the selected diagnostic filter.

In some implementations, the user interface of the auscultation system can include a display, for example, a touch sensitive display. In some cases, the display is capable of providing a list of pathologies (i.e., aortic stenosis, mitrial stenosis, etc.), a list of auscultatory findings, a list of associated pathologies for a particular auscultatory findings, and/or a list of characteristics of a particular pathology (i.e. severity levels, auscultatory findings, etc). In some embodiments, a user can select a diagnostic filter from the list, by pressing a button or a position on the display, for example, depending on the type of display and input methods.

The present invention should not be considered limited to the particular examples and embodiments described above, as such embodiments are described in detail to facilitate explanation of various aspects of the invention. Rather the present invention should be understood to cover all aspects of the invention, including various modifications, equivalent processes, and alternative devices falling within the spirit and scope of the invention as defined by the appended claims.

What is claimed is:

1. An auscultation system comprising:
a sensor configured to detect acoustic signals from human body and generate medical measurement signals based on the detected acoustic signals;
a digital filter, wherein the digital filter is capable of filtering the medical measurement signals with a plurality of stethoscope simulation filters, each stethoscope simulation filter operable to provide a transfer function, the transfer function resulting in a frequency response of the auscultation system that is psychoacoustically equivalent to a frequency response of a mechanical stethoscope; and
a user interface configured to accept input from a user, wherein the user interface is further configured to allow a user to select a stethoscope simulation filter from the plurality of stethoscope simulation filters and the digital filter is configured to filter the medical measurement signals with the selected stethoscope simulation filter.

2. The auscultation system of claim 1, wherein the digital filter comprises a microprocessor configured to apply digital filtering to the processed medical measurement signals.

3. The auscultation system of claim 1, further comprising:
a transmitter coupled to the signal circuit and configured to send the medical measurement signals via a communication network; and
a receiver coupled to the digital filter and configured to receive the medical measurement signals via the communication network.

4. The auscultation system of claim 1, wherein the transfer function provided by each stethoscope simulation filter results in a frequency response of the auscultation system within a 3 dB deviation from a frequency response of a mechanical stethoscope for each ⅓ octave frequency band in a frequency range of 20 Hz to 1200 Hz.

5. The auscultation system of claim 1, wherein the digital filter is further capable of filtering the medical measurement signals with one or more headset filters, each headset filter operable to provide a transfer function to compensate for a characteristic frequency response of a particular headset, and
wherein the user interface is further configured to allow a user to select a headset filter from the one or more headset filters and the digital filter is configured to filter the medical measurement signals with the selected headset filter.

6. The auscultation system of claim 1, wherein the digital filter is further capable of filtering the medical measurement signals with one or more diagnostic filters, each diagnostic filter operable to provide a band-pass filter to filter the medical measurement signals at one or more frequency ranges appropriate to a particular diagnosis, and
wherein the user interface is further configured to allow a user to select a diagnostic filter from the one or more diagnostic filters and the digital filter is configured to filter the medical measurement signals with the selected diagnostic filter.

7. The auscultation system of claim 1, wherein the deviation in frequency response is determined using an air-coupled test method.

8. The auscultation system of claim 1, wherein the mechanical stethoscope is one of a LITTMANN MASTER CARDIOLOGY stethoscope.

9. An auscultation system comprising:
a sensor configured to detect acoustic signals from human body and generate medical measurement signals based on the detected acoustic signals;
a digital filter, wherein the digital filter is capable of filtering the medical measurement signals with one or more headset filters, each headset filter operable to provide a transfer function to compensate for a characteristic frequency response of a particular headset; and
a user interface configured to accept input from a user, wherein the user interface is further configured to allow a user to select a headset filter from the one or more headset filters and the digital filter is configured to filter the medical measurement signals with the selected headset filter.

10. The auscultation system of claim 9, wherein the digital filter is a microprocessor configured to apply digital filtering to the processed medical measurement signals.

11. The auscultation system of claim 9, further comprising:
a transmitter coupled to the signal circuit and configured to send the medical measurement signals via a communication network; and
a receiver coupled to the digital filter and configured to receive the medical measurement signals via the communication network.

12. The auscultation system of claim 9, wherein the digital filter is further capable of filtering the medical measurement signals with one or more stethoscope simulation filters, each stethoscope simulation filter operable to provide a transfer function, the transfer function resulting in a frequency response of the auscultation system that is psychoacoustically equivalent to a frequency response of a mechanical stethoscope, and
wherein the user interface is further configured to allow a user to select a stethoscope simulation filter from the one or more stethoscope simulation filters and the digital filter is configured to filter the medical measurement signals with the selected stethoscope simulation filter.

13. The auscultation system of claim 12, wherein the transfer function provided by each stethoscope simulation filter results in a frequency response of the auscultation system within a 3 dB deviation from a frequency response of a mechanical stethoscope for each ⅓ octave frequency band in a frequency range of 20 Hz to 1200 Hz.

14. The auscultation system of claim 9, wherein the digital filter is further capable of filtering the medical measurement signals with one or more diagnostic filters, each diagnostic filter operable to provide a band-pass filter to filter the medical measurement signals at one or more frequency ranges appropriate to a particular diagnosis, and
wherein the user interface is further configured to allow a user to select a diagnostic filter from the one or more diagnostic filters and the digital filter is configured to filter the medical measurement signals with the selected diagnostic filter.

15. An auscultation system comprising:
a sensor configured to detect acoustic signals from human body and generate medical measurement signals based on the detected acoustic signals;
a digital filter, wherein the digital filter is capable of filtering the medical measurement signals with one or more diagnostic filters, each diagnostic filter operable to provide a band-pass filter to filter the medical measurement signals at one or more frequency ranges appropriate to a particular diagnosis; and
a user interface configured to accept input from a user, wherein the user interface is further configured to allow a user to select a diagnostic filter from the one or more diagnostic filters and the digital filter is configured to filter the medical measurement signals with the selected diagnostic filter.

16. The auscultation system of claim 15, wherein the digital filter comprises a microprocessor configured to apply digital filtering to the processed medical measurement signals.

17. The auscultation system of claim 15, further comprising:
a transmitter coupled to the signal circuit and configured to send the medical measurement signals via a communication network; and
a receiver coupled to the digital filter and configured to receive the medical measurement signals via the communication network.

18. The auscultation system of claim 15, wherein the digital filter is further capable of filtering the medical measurement signals with one or more stethoscope simulation filters, each stethoscope simulation filter operable to provide a transfer function, the transfer function resulting in a frequency response of the auscultation system that is psychoacoustically equivalent to a frequency response of a mechanical stethoscope, and
wherein the user interface is further configured to allow a user to select a stethoscope simulation filter from the one or more stethoscope simulation filters and the digital filter is configured to filter the medical measurement signals with the selected stethoscope simulation filter.

19. The auscultation system of claim 18, wherein the transfer function provided by each stethoscope simulation filter results in a frequency response of the auscultation system within a 3 dB deviation from a frequency response of a mechanical stethoscope for each ⅓ octave frequency band in a frequency range of 20 Hz to 1200 Hz.

20. The auscultation system of claim 15, wherein the digital filter is further capable of filtering the medical measurement signals with one or more headset filters, each headset filter operable to provide a transfer function to compensate for a characteristic frequency response of a particular headset, and
wherein the user interface is further configured to allow a user to select a headset filter from the one or more headset filters and the digital filter is configured to filter the medical measurement signals with the selected headset filter.
